(19) United States
(12) Patent Application Publication
(10) Pub. No.: US 2025/0164431 A1
(43) Pub. Date: May 22, 2025

(54) GENERATING A SUPPLY VOLTAGE FOR A RESISTIVE SENSOR ELEMENT

(71) Applicant: Infineon Technologies AG, Neubiberg (DE)

(72) Inventors: Michael KANDLER, Sauerlach (DE); Matthias EBERL, Taufkirchen (DE)

(21) Appl. No.: 18/943,330

(22) Filed: Nov. 11, 2024

(30) Foreign Application Priority Data
Nov. 17, 2023 (DE) 102023211448.8

(51) Int. Cl. G01N 27/18; G01N 33/00

(52) U.S. Cl. CPC G01N 27/18; G01N 33/0073

(57) ABSTRACT

A circuit arrangement for operating a resistive sensor element is described. According to an example implementation, the circuit arrangement includes a first supply terminal and a second supply terminal, between which is applied a supply voltage during operation, and a sensor circuit having at least one resistive sensor element. The sensor circuit has a first circuit node and a second circuit node for applying a sensor voltage, wherein the first circuit node is connected to the first supply terminal. The circuit arrangement further includes a sensor supply circuit, which is configured to electrically couple, during a measurement time interval, a charged capacitor to the second circuit node in such a way that the sensor voltage between the first circuit node and the second circuit node is greater than the supply voltage.

### CROSS-REFERENCE TO RELATED APPLICATION

This application claims priority to Germany Patent Application No. 102023211448.8 filed on Nov. 17, 2023, the content of which is incorporated by reference herein in its entirety.

### TECHNICAL FIELD

The present description relates to the field of sensors, in particular to generating a supply voltage for one or more resistive sensor elements.

### BACKGROUND

There are numerous sensors having resistive sensor elements, the electrical resistance of which depends directly or indirectly on the physical quantity to be measured. An example thereof are TC sensors (thermal conductivity sensors) for measuring the thermal conductivity of gases. In this type of sensor, a resistance (sensor element) is heated by applying a voltage. The resistance is exposed to an analysis gas. The voltage applied to the sensor element causes a sensor current to flow, resulting in a certain power dissipation in the sensor element; on account of the power dissipation, the sensor element (e.g., a resistance) heats up to a certain temperature above the ambient temperature. At this temperature, the total heat lost from the resistance equals the electrical energy consumed. The heat loss largely depends on the thermal conductivity of the analysis gas. The higher the thermal conductivity of the gas, the higher the cooling action and the lower the temperature (and the greater the electrical conductivity) of the sensor element given a constant voltage. The greater the electrical conductivity of the resistance of the sensor element, the higher the current. The current is thus a measure of the thermal conductivity of the analysis gas.

The sensitivity of such a TC sensor increases with the supply voltage. The output signal from the sensor has an approximately cubic dependency on the applied supply voltage. The supply voltage for the sensor is not applied continuously but at regular intervals (periodically or from time to time) in order to lower the average power consumption and to increase the operating life of the sensor.

In many applications, the operation of the sensor is controlled by an integrated control circuit, for example by a microcontroller. Such a control circuit—like many other integrated circuits—can be operated using a relatively low supply voltage, for instance 3.3 Volts. This voltage can be too low, however, for resistive sensor elements, in particular for the aforementioned TC sensors, to achieve a sufficient sensor sensitivity.

The inventors have set themselves the object of improving existing concepts for operating resistive sensor elements, in particular with regard to generating the sensor supply voltage.

### SUMMARY

The aforementioned object is achieved by the circuit arrangement as claimed in claim 1 and by the method as claimed in claim 11. The subject matter of the dependent patent claims contains various example implementations and further developments.

A circuit arrangement for operating a resistive sensor element is described below. According to an example implementation, the circuit arrangement includes a first supply terminal and a second supply terminal, between which is applied a supply voltage during operation, and a sensor circuit having at least one resistive sensor element. The sensor circuit has a first circuit node and a second circuit node for applying a sensor voltage, wherein the first circuit node is connected to the first supply terminal. The circuit arrangement further includes a sensor supply circuit, which is configured to couple electrically, during a measurement time interval, a charged capacitor to the second circuit node in such a way that the sensor voltage between the first circuit node and the second circuit node is greater than the supply voltage.

In addition, a method is described for operating a sensor circuit having at least one resistive sensor element, wherein the sensor circuit has a first circuit node and a second circuit node for applying a sensor voltage. According to an example implementation, the method includes charging a capacitor by coupling the capacitor to a voltage supply, which provides a supply voltage between a first supply terminal and a second supply terminal, wherein the first circuit node of the sensor circuit is connected to the first supply terminal. The method further includes coupling the charged capacitor to the second circuit node of the sensor circuit during a measurement time interval in such a way that the resultant sensor voltage between the first circuit node and the second circuit node of the sensor circuit is greater than the supply voltage.

### BRIEF DESCRIPTION OF THE DRAWINGS

Example implementations are described in greater detail below with reference to figures. The representations are not necessarily to scale, and the example implementations are not restricted just to the represented aspects. Rather, emphasis is placed on presenting the principles underlying the example implementations. In the figures:

FIG. 1 illustrates the construction of a sensor element of a TC sensor using micromechanical structures;

FIG. 2 shows a bridge circuit (resistance bridge) that can be used in a TC sensor;

FIG. 3 is a graph for illustrating the dependency of the sensor sensitivity on the sensor voltage;

FIG. 4 shows as a first example implementation a circuit arrangement having a supply circuit for generating a sensor voltage for a sensor having resistive sensor elements;

FIGS. 5A-5C contain a plurality of timing diagrams for illustrating the operation of the circuit arrangement from FIG. 4;

FIG. 6 shows a further example implementation of the supply circuit from FIG. 4 in greater detail;

FIG. 7 shows an alternative (equivalent) implementation of the example from FIG. 4;

FIGS. 8 and 9 show additions/modifications to the example from FIG. 4, in which the sensor voltage across the bridge is measured in addition to the bridge output voltage;

FIG. 10 is a graph for illustrating a measurement process.

### DETAILED DESCRIPTION

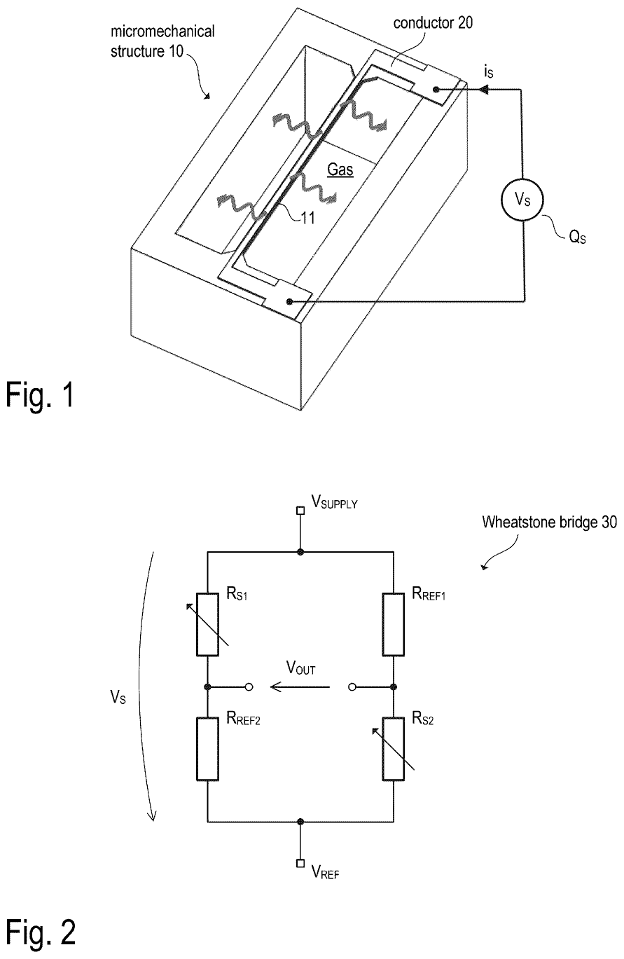

FIG. 1 shows a micromechanical structure of a resistive sensor element of a TC sensor. The micromechanical structure can be integrated in a silicon chip, which in turn is mounted in a chip housing (package). Various methods for fabricating integrated micro-electromechanical systems (MEMS) are known per se and therefore are not discussed further here.

According to the example shown in FIG. 1, the micromechanical structure 10 forms a frame, which can be rectangular in shape, for example. The shape of the frame is not essential, however. Across the frame (like a “bridge” from one side of the frame to an opposite side) runs an electrical conductor 20, which can be in the form of a strip conductor, for instance. The conductor 20 can consist of metal (e.g., aluminum) or polysilicon. In the case of a metal conductor, this can be mounted on a crosspiece made of silicon, which connects two opposite sides of the frame 10.

The micromechanical structure 10 forms a cavity, in which can be situated the analysis gas mentioned in the introduction. In practice, the analysis gas is often a mixture of a plurality of gaseous materials, such as air for instance. FIG. 1 also shows a voltage source Q_{S}, which generates the sensor voltage V_{S}. The resultant sensor current is depends on the resistance R_{S} (electrical conductivity σ_{S}=R_{S}⁻¹) of the sensor element. The power P_{S} converted into heat in the sensor element is equal to V_{S}²·θ_{S}=i_{S}²·R_{S}. In thermal equilibrium (e.g., when the conductor 20 is at a constant temperature), the heat removed per unit of time (cooling capacity) equals the electrical power V_{S}²·σ_{S}. The cooling capacity is in turn higher for a higher thermal conductivity κ of the analysis gas. Therefore, the higher the thermal conductivity κ of the analysis gas, the lower the temperature T_{S} of the conductor 20. A lower temperature T_{S} of the conductor 20, however, results in a higher electrical conductivity σ_{S} (because the temperature coefficient of the resistance R_{S} is positive), e.g., σ_{S}=σ_{S}(κ) is a function of the thermal conductivity κ. Hence also the sensor current is depends on the thermal conductivity κ, because is =V_{S}·θ_{S}(κ). A higher thermal conductivity κ results in a lower temperature T_{S} and hence in a higher electrical conductivity σ_{S} and a higher sensor current i_{S}.

As in many other resistive sensors, the resistive sensor elements of FIG. 1 can be connected to form a bridge circuit (also called a Wheatstone bridge). FIG. 2 shows an example. According to this example, the bridge circuit comprises two voltage dividers, which are connected between a first bridge supply node (to which is applied the supply voltage V_{SUPPLY}) and a second bridge supply node (to which is applied e.g., a reference or ground potential V_{REF}). Two of the four bridge resistances are sensor elements as shown in FIG. 1 and are denoted by R_{S1} and R_{S2}. The two other resistances are reference resistances R_{REF1} and R_{REF2}. Assuming R_{REF1}=R_{REF2}=R and R_{S1}=R_{S2}=R+ΔR, and making the approximation 2R+ΔR≈2R (because ΔR<<R), then it holds that the bridge output voltage V_{OUT}=(V_{SUPPLY}−V_{REF})·ΔR/2, where ΔR denotes the difference of the sensor resistance from the known, nominal resistance value R. In many known sensor circuits, the reference potential V_{REF} equals the ground potential, namely 0 Volts.

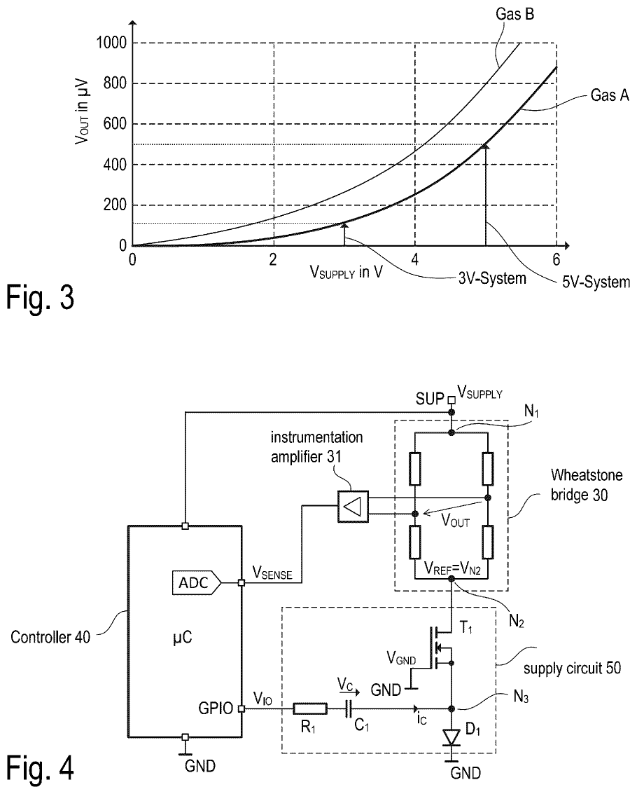

The graph of FIG. 3 illustrates the dependency of the bridge output voltage VOUT on the bridge supply voltage V_{SUPPLY} (assuming V_{REF}=0V). The two curves shown relate to different gaseous substance mixtures, which are labeled as “Gas A” and “Gas B”. It is apparent that the bridge output voltage VOUT increases disproportionately with the bridge supply voltage V_{SUPPLY}, because at a higher bridge supply voltage V_{SUPPLY}, the current through the sensor elements, and hence their temperature T_{S}, also rises. If the voltage V_{SUPPLY} is increased by two thirds (e.g., from 3V by about 67% to 5V), the bridge output voltage VOUT rises by significantly more than 67%; in the examples shown by several hundred percent.

Sensors usually comprise in addition to the sensor elements also circuits for driving the sensor elements and for (post) processing the sensor signal (e.g., the bridge output voltage V_{OUT}). These tasks can be performed by control circuits, which can contain a microcontroller, for example. The control circuit (microcontroller) can have, for example, an analog-to-digital converter, which is configured to digitize the output voltage of a sensor element or the bridge output voltage V_{OUT} from a resistance bridge (cf. FIG. 2). Modern digital circuits such as microcontrollers work with relatively small supply voltages such as 3.3 Volts, for example. As explained above with reference to FIG. 3, however, a higher supply voltage is advantageous for resistive sensors (for which the sensor elements are arranged in a resistance bridge, for example) and in particular for TC sensors.

For these reasons, known sensor concepts need two different supply voltages, where, for example, a linear voltage regulator is used to generate a 3.3V supply voltage for the microcontroller from a 6V supply voltage applied to the resistance bridge, which results in unwanted losses in the voltage regulator. It is also possible to use a DC/DC switching converter instead of a voltage regulator in order to generate, for example, a 3.3V supply voltage from a 6V supply voltage, or vice versa. Such DC/DC switching converters, for instance boost converters or buck converters, are complex, however, and require an inductance, which cannot be integrated in a chip.

The circuit arrangement shown in FIG. 4 comprises a resistance bridge 30, a control circuit 40 (microcontroller) having an analog-to-digital converter for digitizing the bridge output voltage V_{OUT}, and a supply circuit, which is configured to provide the bridge circuit 30 temporarily (during a measurement time interval) with a supply voltage V_{SUPPLY}−V_{REF} which is greater than the supply voltage V_{SUPPLY} that also supplies the control circuit 40.

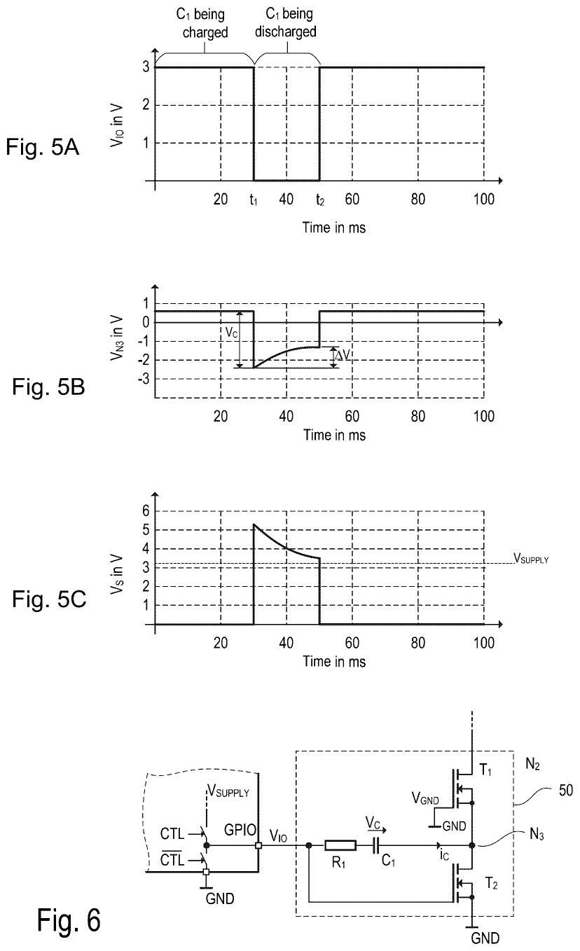

In the example shown in FIG. 4, the supply voltage equals 3 Volts (V_{SUPPLY}=3V). The supply voltage V_{SUPPLY} is taken to the control circuit 40 and the first bridge supply node N₁. The second bridge supply node N₂ is at a reference voltage V_{N2}=V_{REF}, which in the examples described here is not constant but provided by a supply circuit 50. The operation of the supply circuit is controlled by the control circuit 40 (microcontroller), wherein the control circuit 40 is configured to apply temporarily (during a measurement time interval) to the bridge supply node N₂ a voltage V_{N2} which lies below the operating voltage range of 0-3V. This means that the voltage V_{N2} is negative during the measurement time interval, and the bridge supply voltage V_{SUPPLY}−V_{N2} is consequently greater than the supply voltage V_{SUPPLY}. The timing diagrams of FIGS. 5A-5C are used below to explain in greater detail the manner of operation of the supply circuit 50.

The supply circuit 50 receives as a control signal a logic signal V_{IO}, which is generated by the microcontroller 40. Standard microcontrollers have digital outputs at which a logic signal can be output. Some microcontrollers have configurable terminals (known as General Purpose IO (GPIO) terminals), which can be configured both as a digital input and as a digital output. Such GPIO terminals can also be used to output the logic signal V_{IO}. The logic signal V_{IO} can assume only two different levels, namely a low level, which equals approximately the ground potential V_{GND}=0V, and a high level, which equals approximately the supply voltage V_{SUPPLY}. In the example shown, the supply circuit 50 has a capacitor C₁, which is connected on one side to a digital output of the microcontroller 40 (e.g., using a resistor R₁ for current limiting) and on the other side to the circuit node N₃. This node N₃ can be connected in turn using a diode D₁ to the ground node GND, which diode D₁ can also be replaced by another component such as a controllable electronic switch, for example (e.g., a transistor or a more complex network). The node N₃ is connected using a further electronic switch T₁ (a MOS transistor in the example shown) to the second bridge supply node N₂. The transistor T₁ also can be replaced by other types of electronic switch or by more complex switching networks.

The microcontroller can indicate the aforementioned measurement time interval by the level of the logic signal V_{IO}. As shown by way of example in diagram of FIG. 5A, the measurement time interval (from time t₁ to time t₂) is defined by a low level (approx. 0V) of the logic signal V_{IO}. Outside the measurement time interval, the logic signal has a high level (approx. 3V in the example shown). As long as the logic signal V_{IO} has a high level, a current i_{C} can flow from the digital output GPIO of the microcontroller 40 through the resistor R₁, the capacitor C₁ and the diode D₁ to the ground node GND and charge the capacitor C₁. The charging process ends when the capacitor voltage V_{C} has reached a stationary value that is substantially equal to the high level of the logic signal minus the forward voltage VF (approx. 0.7V) of the diode D₁ (e.g., V_{C}≈V_{SUPPLY}−V_{F}). Immediately before the time t₁, the capacitor voltage V_{C} is practically constant, and the voltage V_{N3} is approximately equal to the voltage drop V_{F} across the diode D₁ (V_{N3}=V_{F}≈0.7V, see diagram of FIG. 5B). If at time t₁ (e.g., at the start of a measurement time interval) the microcontroller 40 sets the level of the logic signal V_{IO} from high to low, the voltage V_{N3} of the node N₃ then falls abruptly from V_{F} to a negative value of approximately −V_{SUPPLY}+V_{F}, so approx. −2.3 Volts in the present example, and the diode D₁ stops conducting. Simultaneously (also at time t₁), the electronic switch/transistor T₁ switches on and connects the node N₃ to the bridge supply voltage N₂, with the result that a voltage V_{SUPPLY}−V_{N3} is applied to the bridge circuit 30 which is greater than the supply voltage V_{SUPPLY} (initially approx. 2. V_{SUPPLY}−V_{F}). The voltage drop across the transistor T₁ is ignored for the sake of simplicity.

During the measurement time interval (between t₁ and t₂), a current flows through the resistance bridge 30 while the capacitor C₁ is discharged. Consequently, the voltage V_{N3} rises during the measurement time interval, and the bridge supply voltage V_{SUPPLY}−V_{N3} falls correspondingly (see diagram of FIG. 5C). The voltage change ΔV during the measurement time interval can be smaller or larger depending on the capacitance of the capacitor C₁. At the end of the measurement time interval (at time t₂), the microcontroller 40 sets the level of the logic signal V_{IO} from low to high, and the capacitor is recharged relatively quickly (not shown in detail in FIGS. 5A-5C). The diode D₁ starts to conduct again, the electronic switch (transistor T₁) switches off, and the voltage V_{SUPPLY}−V_{N2} across the bridge circuit 30 falls to zero. During the measurement time interval, the microcontroller 40 can sample and digitize the variation in the bridge output voltage V_{OUT} (see also FIG. 10). From the corresponding digital values, the microcontroller 40 can determine the information being sought (e.g., the thermal conductivity of the analysis gas in the case of a TC sensor), For this purpose, the microcontroller can use calibration data and/or characteristic curves stored in a memory.

The circuit shown in FIG. 4 is merely an example. The example implementations described here relate generally to a circuit arrangement for operating a sensor. In a general example, the circuit arrangement is supplied by a supply voltage V_{SUPPLY}. For this purpose, the circuit arrangement has a first supply terminal and a second supply terminal GND, between which the supply voltage V_{SUPPLY} is applied during operation. In addition, the circuit arrangement has a sensor circuit 30 having at least one resistive sensor element (cf. FIG. 2, resistances R_{S1}, R_{S2}). The sensor circuit 30 (e.g., a Wheatstone bridge) is supplied by a sensor voltage V_{S}. The sensor circuit 30 therefore has a first circuit node N₁ and a second circuit node N₂, between which the sensor voltage V_{S} is applied during a measurement (during a measurement time interval). The first circuit node N₁ of the sensor circuit is connected to the first supply terminal (see FIG. 4). The circuit arrangement further comprises a sensor supply circuit 50. This is configured to couple electrically during a measurement (during a measurement time interval) a previously charged capacitor C₁ to the second circuit node N₂ in such a way (using the transistor T₁ in the examples shown) that the sensor voltage V_{S} (which is applied between the first circuit node N₁ and the second circuit node N₂) is greater than the supply voltage V_{SUPPLY}. In other words, the capacitor voltage V_{C} of the charged capacitor C₁ is superimposed (added) onto the supply voltage V_{SUPPLY}. The sensor voltage V_{S} is thus equal—at least temporarily—to approximately V_{SUPPLY}+V_{C}.

FIG. 6 shows a further example of the supply circuit 50, and also illustrates the manner of operation of the digital output GPIO of the microcontroller in greater detail. According to FIG. 6, the microcontroller 40 contains a driver, which outputs the logic signal V_{IO} to the digital output GPIO. In the example shown, the driver comprises two electronic switches: a high-side switch and a low-side switch. The high-side switch couples the output GPIO (e.g., an output pin of the microcontroller 40) to the supply voltage V_{SUPPLY}, and the low-side switch couples the output GPIO to the ground potential V_{GND} at the second supply node GND. The high-side switch is driven by the Boolean signal CTL, and the low-side switch is driven by the inverse signal CTL. In practice, the output stage of the driver can be implemented as a CMOS inverter, for example. The signal CTL can be represented, for example, by a bit in a register that a processor of the microcontroller 40 can set (to “1”) and reset (to “0”). In other words, the signal V_{IO} has either a high level, which equals approximately V_{SUPPLY}, or a low level, which equals approximately V_{GND}.

In other respects, the example from FIG. 6 corresponds to the supply circuit 50 of FIG. 4, with the diode D₁ replaced by a second transistor T₂, the control electrode (gate) of which is also connected to the digital output GIPO. The control electrode of the transistor T₁ is connected to the ground node GND. When a measurement is not taking place, the microcontroller 40 outputs a high level as the logic signal V_{IO}. Hence the transistor T₂ switches on and the transistor T₁ switches off (because the gate-source voltage is approximately zero). The voltage drop across the series circuit R₁-C₁ is consequently approximately equal to the high level of the signal V_{IO} (e.g., V_{IO}≈V_{SUPPLY}), and the capacitor C₁ is charged up until the capacitor voltage V_{C} equals the high level of the logic signal V_{IO} (e.g., V_{C}≈V_{IO}≈V_{SUPPLY}). The resistor R₁ is optional and is used only for current limiting.

During a measurement (in the measurement time interval), the microcontroller 40 outputs as the logic signal V_{IO} a low level (around V_{GND}=0V). As a result, the transistor T₂ switches off, and the electrical potential at the node N₃ is reduced from about 0V to a value of approximately V_{N3}=−V_{C}. In the example from FIG. 4, the diode D₁ is non-conducting because of the negative potential at the node N₃. The negative electrical potential V_{N3} at the node N₃ also causes the transistor T₁ to switch on (positive gate-source voltage at T₁), and therefore also the second circuit node N₂ of the sensor circuit 30 (bridge circuit) has the electrical potential V_{N2}=V_{N3}=−V_{C}, which results in the sensor voltage V_{S} across the resistance bridge 30 being equal to approximately the sum V_{SUPPLY}+V_{C} (V_{S}=V_{SUPPLY}−V_{N2}=V_{SUPPLY}−(−V_{C})=V_{SUPPLY}+V_{C}). During the measurement, the sensor voltage V_{S} is therefore greater than the supply voltage V_{SUPPLY}, which can result in a higher sensitivity, as explained in the introduction (cf. FIG. 3). During the measurement, the capacitor C₁ discharges and the capacitor voltage V_{C} falls. The sensor voltage V_{S} (bridge voltage) is not constant during a measurement, as illustrated in diagram of FIG. 5C.

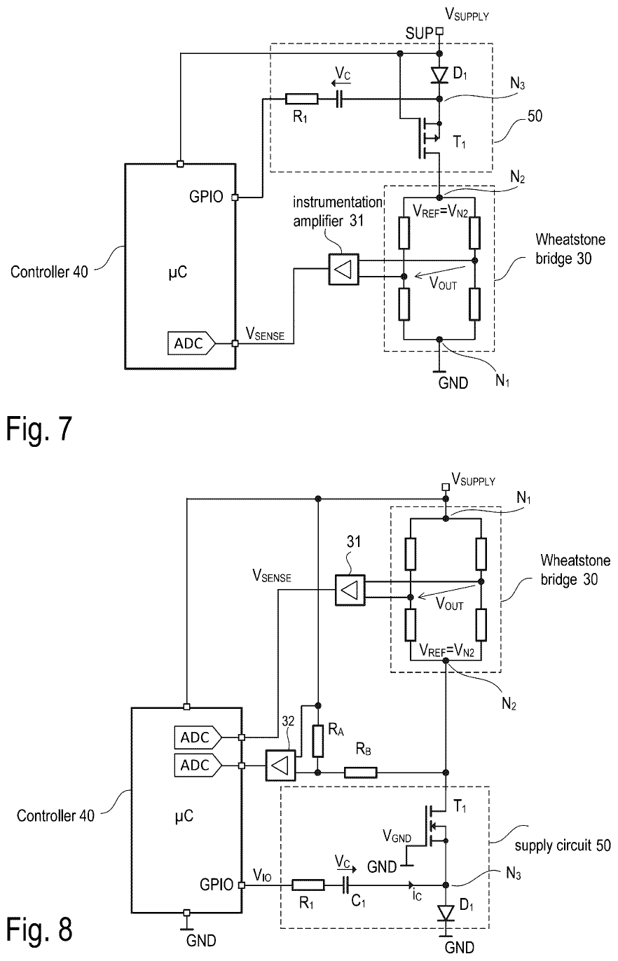

FIG. 7 shows an alternative (although functionally equivalent) implementation of the example from FIG. 4. It can also be the that the circuit in FIG. 7 is the complement of the circuit in FIG. 4. Sensor circuit 30 and supply circuit 50 are effectively reversed, with the polarity of diodes changed, logic signals inverted and n-channel transistors replaced by p-channel transistors (and vice versa). The operation of the circuit arrangement does not change. To charge the capacitor C₁, the logic signal V_{IO} now has a low level. The diode D₁ is therefore conducting, the (p-channel) transistor T₁ is off, and current can flow from the voltage supply through the capacitor C₁, the resistor R₁ to the digital output GPIO, thereby charging the capacitor. For a measurement, the microcontroller 40 outputs a high level as the logic signal V_{IO}. This raises the electrical potential V_{N3} at the node N₃ above the supply voltage V_{SUPPLY} (V_{N3}=V_{SUPPLY}+V_{C}). As a result, the diode D₁ stops conducting and the transistor T₁ switches on, causing the electrical potential V_{N2} at the node N₂ to rise (V_{N2}≈V_{N3}=V_{SUPPLY}+V_{C}). The resistance bridge 30 “sees” a sensor voltage V_{S}=V_{N2}−V_{N1}=V_{N2}, which is greater than the supply voltage V_{SUPPLY} (analogous to the examples from FIGS. 4 and 6).

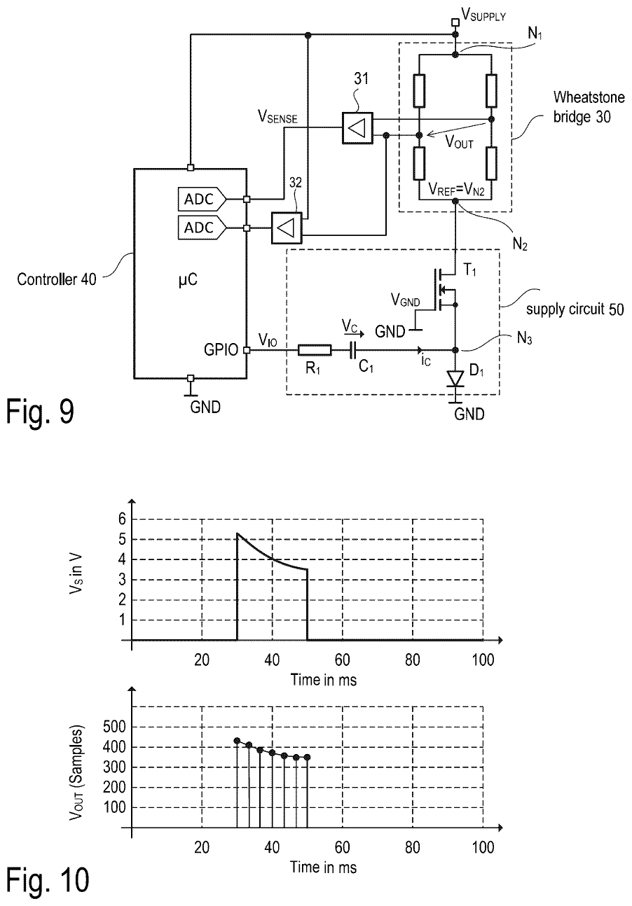

FIGS. 8 and 9 show additions/modifications to the example from FIG. 4, in which the sensor voltage V_{S} across the bridge is also measured indirectly in addition to the bridge output voltage. In the example shown in FIG. 7, a voltage divider containing the resistors RA and RB is connected in parallel with the sensor element 30 (Wheatstone bridge). The voltage across the resistor R_{A} equals V_{S}·R_{A}/(R_{A}+R_{B}). If both resistors are the same (R_{A}−R_{B}), then the voltage across the resistor R_{A} equals V_{S}/2. This voltage can be taken to a differential amplifier 32 (e.g., having a gain of one), and the output signal from the amplifier 32 can be taken to the analog input of an analog-to-digital converter contained in the microcontroller 40, which converter digitizes (samples and quantizes) the voltage V_{S}·R_{A}/(R_{A}+R_{B}). The corresponding digital values represent the sensor voltage V_{S} during the measurement time interval. In other respects the example from FIG. 8 is identical to the example from FIG. 4, and reference is made to the associated description given above.

The example from FIG. 9 is very similar to the example from FIG. 7, except that part of the resistance bridge is used instead of the voltage divider (resistors R_{A} and R_{B}). Assuming R_{S1}=R+ΔR and R_{REF1}=R, and making the approximation ΔR/R<<1 (ΔR is negligibly small compared with R), the differential amplifier 32 “sees”, as in the example from FIG. 7, half the sensor voltage V_{S}/2 if the voltage across the resistance R_{S1} is taken to the amplifier 32 (see FIG. 7 in conjunction with FIG. 2). In other respects, the example from FIG. 9 is identical to the example from FIG. 8.

FIG. 10 shows in a timing diagram a measurement time interval (analogous to FIGS. 5A-5C). The top diagram of FIG. 10 shows the sensor voltage V_{S} (V_{S}=V_{SUPPLY}−V_{N2} in the example in FIG. 4) and the bottom diagram of FIG. 10 shows the corresponding bridge output voltage V_{OUT} and the corresponding digital values (samples), which the control circuit 40 (e.g., microcontroller) generates using an analog-to-digital converter. Since the capacitor C₁ (see e.g., FIG. 4 or FIG. 7) discharges continuously during a measurement (which lasts 10 ms in the example from FIG. 10), the sensor voltage V_{S} and hence also the bridge output voltage V_{OUT} are not constant. Thus, in a measurement time interval (measurement cycle), a multiplicity of individual measured values V_{OUT} are captured at different sensor voltages V_{S}. From the (digitized) measured values, the microcontroller 40 can determine the desired information (e.g., the thermal conductivity of the analysis gas in the case of a TC sensor). For this purpose, the microcontroller can use calibration data and/or characteristic curves stored in a memory, and/or post-process the measured value digitally. The capture of a multiplicity of measured values at different sensor voltages V_{S} also allows the correction of cross-sensitivities to other physical parameters (e.g., humidity), which can influence the output voltage V_{OUT}.

In the depicted example implementations, the control circuit 40 is a microcontroller having at least one processor, which is configured to execute software instructions stored in a memory in order to perform the functions described here, for instance controlling the measurement time intervals (measurement cycles) by outputting the signal V_{IO} to a digital output, and controlling the operation of the analog-to-digital converter (see e.g., FIG. 4). In many microcontrollers are integrated one or more analog-to-digital converters. It is also possible to use separate analog-to-digital converters, however. In the examples from FIGS. 8 and 9, a multiplexer and a single analog-to-digital converter can also be used instead of two analog-to-digital converters, in which case the multiplexer is used with the analog-to-digital converter to digitize the two analog measured values one after the other. The control circuit 40 need not necessarily have a processor for executing software instructions, however, but can also contain a one-time programmable (OTP) or hard-wired digital circuit which essentially provides the same function.

The functionality of the above-described example implementations is implemented by a method for operating a sensor circuit having at least one resistive sensor element and also a first circuit node and a second circuit node for applying a sensor voltage. According to the examples described here, the method comprises charging a capacitor (cf. e.g., FIG. 4 or FIG. 6, capacitor C₁) by coupling the capacitor to a voltage supply, which provides a supply voltage between a first supply terminal and a second supply terminal (cf. FIG. 4, supply terminals SUP and GND), wherein the first circuit node of the sensor circuit is connected to the first supply terminal. The method further comprises coupling the charged capacitor to the second circuit node of the sensor circuit during a measurement time interval in such a way that the resultant sensor voltage between the first circuit node and the second circuit node of the sensor circuit is greater than the supply voltage (cf. FIGS. 5A-5C, V_{S} is greater than V_{SUPPLY} because V_{N3} becomes negative).

According to an example implementation, an output voltage of the sensor circuit can be measured during the measurement time interval (e.g., using an analog-to-digital converter, see e.g., FIGS. 4 and 7-9). The measurement time interval can be indicated by a logic signal, for instance generated by a control circuit (e.g., a microcontroller). According to the example implementations depicted here, the at least one resistive sensor element can be part of a Wheatstone bridge. The concepts described here, however, do not rely on a resistance bridge, and can also be applied to other resistive sensors. In some example implementations, also the sensor voltage can be measured during the measurement time interval, during which measurement time interval the capacitor discharges and hence the sensor voltage reduces.

By digitizing (sampling and quantizing) both the output voltage of the sensor circuit and the sensor voltage, which supplies the sensor circuit during the measurement time interval, a multiplicity of measured values can be captured at different sensor voltages. In particular in the case of TC sensors, digital post-processing of the measured values allows correction of cross-sensitivities, for example a cross-sensitivity of the sensor element to humidity.

### Aspects

The following provides an overview of some Aspects of the present disclosure:

Aspect 1: A circuit arrangement which has: a first supply terminal and a second supply terminal, between which a supply voltage is applied during operation; a sensor circuit having at least one resistive sensor element, wherein the sensor circuit has a first circuit node and a second circuit node for applying a sensor voltage, and wherein the first circuit node is connected to the first supply terminal; and a sensor supply circuit, which is configured to electrically couple, during a measurement time interval, a charged capacitor to the second circuit node in such a way that the sensor voltage between the first circuit node and the second circuit node is greater than the supply voltage.

Aspect 2: The circuit arrangement as recited in Aspect 1, wherein the charged capacitor is connected, during the measurement time interval, in such a way between the second supply terminal and the second circuit node of the sensor circuit that the sensor voltage is substantially equal to a sum of the supply voltage and a capacitor voltage across the charged capacitor.

Aspect 3: The circuit arrangement as claimed in any of Aspects 1-2, wherein the sensor supply circuit includes an electronic switch, which is configured to connect the charged capacitor to the second circuit node during the measurement time interval based on a logic signal, which indicates the measurement time interval.

Aspect 4: The circuit arrangement as recited in Aspect 3, wherein the sensor supply circuit has a further electronic switch, which is configured to disconnect the charged capacitor from the second supply terminal during the measurement time interval.

Aspect 5: The circuit arrangement as claimed in any of Aspects 1-4, wherein the at least one resistive sensor element of the sensor circuit is part of a Wheatstone bridge, which is supplied in the measurement time interval by the sensor voltage.

Aspect 6: The circuit arrangement as claimed in any of Aspects 1-5, wherein the at least one resistive sensor element is a micro-electromechanical system (MEMS) integrated in a chip, and wherein an electrical resistance of the MEMS depends on a thermal conductivity of an analysis gas surrounding the MEMS.

Aspect 7: The circuit arrangement as claimed in any of Aspects 1-6, further comprising: a control circuit comprising at least one analog-to-digital converter configured to digitize an output voltage of the sensor circuit during the measurement time interval.

Aspect 8: The circuit arrangement as recited in Aspect 7, wherein the control circuit is further configured to digitize, during the measurement time interval, a voltage that is proportional to the sensor voltage.

Aspect 9: The circuit arrangement as recited in Aspect 3, further comprising: a control circuit configured to generate the logic signal, wherein a signal level of the logic signal indicates the measurement time interval.

Aspect 10: The circuit arrangement as recited in Aspect 9, wherein the control circuit is supplied by the supply voltage.

Aspect 11: A method for operating a sensor circuit, which has at least one resistive sensor element, wherein the sensor circuit has a first circuit node and a second circuit node for applying a sensor voltage, the method comprising: charging a capacitor by coupling the capacitor to a voltage supply, which provides a supply voltage between a first supply terminal and a second supply terminal, wherein the first circuit node of the sensor circuit is connected to the first supply terminal; and coupling the charged capacitor to the second circuit node of the sensor circuit during a measurement time interval in such a way that a resultant sensor voltage between the first circuit node and the second circuit node is greater than the supply voltage.

Aspect 12: The method as recited in Aspect 11, further comprising: measuring an output voltage of the sensor circuit during the measurement time interval.

Aspect 13: The method as recited in Aspect 12, further comprising: generating a logic signal that indicates the measurement time interval.

Aspect 14: The method as claimed in any of Aspects 11-13, wherein the at least one resistive sensor element is part of a Wheatstone bridge.

Aspect 15: The method as claimed in any of Aspects 11-14, further comprising: measuring the sensor voltage during the measurement time interval, wherein the capacitor is configured to discharge during the measurement time interval such that the sensor voltage reduces.

Aspect 16: A system configured to perform one or more operations recited in one or more of Aspects 1-15.

Aspect 17: An apparatus comprising means for performing one or more operations recited in one or more of Aspects 1-15.

Aspect 18: A non-transitory computer-readable medium storing a set of instructions, the set of instructions comprising one or more instructions that, when executed by a device, cause the device to perform one or more operations recited in one or more of Aspects 1-15.

Aspect 19: A computer program product comprising instructions or code for executing one or more operations recited in one or more of Aspects 1-15.

1. A circuit arrangement which has:
a first supply terminal and a second supply terminal, between which a supply voltage is applied during operation;
a sensor circuit having at least one resistive sensor element, wherein the sensor circuit has a first circuit node and a second circuit node for applying a sensor voltage, and wherein the first circuit node is connected to the first supply terminal; and
a sensor supply circuit, which is configured to electrically couple, during a measurement time interval, a charged capacitor to the second circuit node in such a way that the sensor voltage between the first circuit node and the second circuit node is greater than the supply voltage.

2. The circuit arrangement as claimed in claim 1, wherein the charged capacitor is connected, during the measurement time interval, in such a way between the second supply terminal and the second circuit node of the sensor circuit that the sensor voltage is substantially equal to a sum of the supply voltage and a capacitor voltage across the charged capacitor.

3. The circuit arrangement as claimed in claim 1, wherein the sensor supply circuit includes an electronic switch, which is configured to connect the charged capacitor to the second circuit node during the measurement time interval based on a logic signal, which indicates the measurement time interval.

4. The circuit arrangement as claimed in claim 3, wherein the sensor supply circuit has a further electronic switch, which is configured to disconnect the charged capacitor from the second supply terminal during the measurement time interval.

5. The circuit arrangement as claimed in claim 1, wherein the at least one resistive sensor element of the sensor circuit is part of a Wheatstone bridge, which is supplied in the measurement time interval by the sensor voltage.

6. The circuit arrangement as claimed in claim 1, wherein the at least one resistive sensor element is a micro-electromechanical system integrated in a chip, and
wherein an electrical resistance of the MEMS depends on a thermal conductivity of an analysis gas surrounding the MEMS.

7. The circuit arrangement as claimed in claim 1, further comprising:
a control circuit comprising at least one analog-to-digital converter configured to digitize an output voltage of the sensor circuit during the measurement time interval.

8. The circuit arrangement as claimed in claim 7, wherein the control circuit is further configured to digitize, during the measurement time interval, a voltage that is proportional to the sensor voltage.

9. The circuit arrangement as claimed in claim 3, further comprising:
a control circuit configured to generate the logic signal, wherein a signal level of the logic signal indicates the measurement time interval.

10. The circuit arrangement as claimed in claim 9, wherein the control circuit is supplied by the supply voltage.

11. A method for operating a sensor circuit, which has at least one resistive sensor element, wherein the sensor circuit has a first circuit node and a second circuit node for applying a sensor voltage, the method comprising:
charging a capacitor by coupling the capacitor to a voltage supply, which provides a supply voltage between a first supply terminal and a second supply terminal, wherein the first circuit node of the sensor circuit is connected to the first supply terminal; and
coupling the charged capacitor to the second circuit node of the sensor circuit during a measurement time interval in such a way that a resultant sensor voltage between the first circuit node and the second circuit node is greater than the supply voltage.

12. The method as claimed in claim 11, further comprising:
measuring an output voltage of the sensor circuit during the measurement time interval.

13. The method as claimed in claim 12, further comprising:
generating a logic signal that indicates the measurement time interval.

14. The method as claimed in claim 11, wherein the at least one resistive sensor element is part of a Wheatstone bridge.

15. The method as claimed in claim 11, further comprising:
measuring the sensor voltage during the measurement time interval,
wherein the capacitor is configured to discharge during the measurement time interval such that the sensor voltage reduces.
